(19) United States
(12) Patent Application Publication
(10) Pub. No.: US 2018/0174677 A1
(43) Pub. Date: Jun. 21, 2018

(54) AUTISM THERAPY MANAGEMENT SYSTEM AND METHOD

(71) Applicant: Centria Healthcare LLC, Novi, MI (US)

(72) Inventors: Scott Barry, Novi, MI (US); Elliot Chocron, Novi, MI (US); Christopher Wilcox, Novi, MI (US)

(21) Appl. No.: 15/825,651

(22) Filed: Nov. 29, 2017

Related U.S. Application Data
(60) Provisional application No. 62/428,343, filed on Nov. 30, 2016.

(51) Int. Cl. G16H 20/70; A61B 5/00; A61B 5/16

(52) U.S. Cl. CPC G16H 20/70; A61B 5/167; A61B 5/4848

(57) ABSTRACT

A system and method is provided for automatically conducting tasks in support of autism therapy sessions for a human subject. The method includes the steps of: conducting a subject assessment to generate assessment results relating to degrees of autism of the human subject; documenting the assessment results electronically; creating an autism therapy plan by inputting the assessment results into a mathematical model; implementing the autism therapy plan by conducting therapy programs; monitoring therapy program results, and creating a revised autism therapy plan which is generated using the therapy program results as an input.

### CROSS-REFERENCE TO RELATED APPLICATION

This application claims priority of U.S. Provisional Patent Application Ser. No. 62/428,343 filed Nov. 30, 2016, which is incorporated herein by reference.

### FIELD OF THE INVENTION

The present invention in general relates to subject care, and in particular to a system and method for managing autism therapy.

### BACKGROUND OF THE INVENTION

Having an autism disorder, or a similar condition, such as Asperger's syndrome, can create challenges for a subject to navigate the world. However, autism therapies have been developed which, among other things, can help to teach life skills and other techniques to an autistic subject to better understand and therefore interface with the larger world.

A number of tools and techniques can be used for therapy of autistic subjects, Asperger's subjects, or, in general, subjects who are on the autism spectrum of social responses. These tools and techniques can include: assessments to determine details of a subject's condition, including whether the subject may be on the autism spectrum; creating an autism therapy plan; and conducting an autism therapy session in which parts of the autism therapy plan are carried out.

One example of autism therapy is called Applied Behavior Analysis therapy, also known as ABA therapy. ABA therapy may be used to teach communication skills, teach life skills, and/or modify problem behavior. The therapy involves breaking down skills into small steps which are easier for autism subjects to understand and therefore learn. ABA therapy can be done either in a home environment or a center environment, and often proves effective in helping autistic subjects acclimate to the larger world.

During ABA therapy, data may be collected on an ongoing basis to measure progress. This data may then be used to modify a therapy plan for the subject, or to create a new therapy plan. The resulting new or modified plan may then be applied in subsequent therapy. Thus, feedback from autism therapy sessions with a subject may be used to help customize a therapy plan for use in future therapy sessions. Traditionally, this data has been collected as hand written notes or audio notes that are highly subjective and difficult to compare between practitioners.

Many components of autism therapy can be complex and cumbersome. Some of this complexity and cumbersomeness is due to the burdensome nature of collecting data or creating documentation by hand, and some of it is because of how effort-intensive it can be to use feedback data from therapy to modify a therapy plan or create a new one, especially when new feedback data is continually being collected. Thus, there exists a need for improved methods for managing and administering autism therapy.

### SUMMARY OF THE INVENTION

A method is provided for using a computing device to automatically conduct tasks in support of autism therapy sessions for a human subject. The method includes creating an autism therapy plan by inputting the assessment results into a mathematical model, implementing the autism therapy plan by conducting therapy programs, monitoring therapy program results, and creating a revised autism therapy plan which is generated using the therapy program results as an input.

A method is provided for using a computing device to on-board and assign clients and staff in support of autism services. The method includes registering through the use of a web portal, verifying payment information through the web portal, creating an agency database, and providing a login link to an administrator. The method further includes accessing the agency database through the login link, adding autism clients and staff to the agency database, assigning staff to autism clients, and creating an autism behavior therapy plan.

A method is provided for using a mobile device to initiate autism therapy sessions. The method includes logging in, selecting a client, selecting a program for use in autism therapy sessions with the client, applying filters to the program, viewing the program, entering a planned session duration, and starting a session.

A method is provided for using a mobile device to conduct trials in autism therapy sessions. The method includes selecting a program, selecting a target, and using logic to automatically determine if the selected target is in probe mode. If the logic determines that the selected target is in probe mode, entering or populating probe details, and recording probe details using electronic documentation, and alternatively, if the logic determines that the selected target is not in probe mode, recording trials using electronic documentation.

A system is provided that includes a computing device adapted to automatically conduct tasks in support of autism therapy sessions for a human subject. The tasks include creating an autism therapy plan by inputting the assessment results into a mathematical model, implementing the autism therapy plan by conducting therapy programs, monitoring therapy program results, and creating a revised autism therapy plan which is generated using the therapy program results as an input.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a flow diagram of a high-level overview of a process for using a computing device to automatically conduct tasks in support of autism therapy sessions for a human subject in accordance with embodiments of the invention;

FIG. 2 is a flow diagram representing possible sub-processes of the steps of “Conduct Subject Assessment” from FIG. 1 in accordance with embodiments of the invention;

FIG. 3 is a flow diagram representing possible sub-processes of the steps of “Create/Update Therapy Plan Using Results” from FIG. 1 in accordance with embodiments of the invention;

FIG. 4 is a flow diagram representing possible sub-processes of the steps of “Implement Plan in Therapy” from FIG. 1 in accordance with embodiments of the invention;

FIG. 5 is a flow diagram representing possible sub-processes of the step of “Work Through Programs” from FIG. 4 in accordance with embodiments of the invention;

FIG. 6 is a flow diagram representing possible sub-processes of the step of “Monitor Plan” from FIG. 1 in accordance with embodiments of the invention;

FIG. 7 shows a representative login screen for a web portal in accordance with embodiments of the invention;

FIG. 8 shows a user account information screen, containing information related to a user's web portal account in accordance with embodiments of the invention;

FIG. 9 shows a client information screen in accordance with embodiments of the invention;

FIG. 10 shows an interface for managing therapy programs in accordance with embodiments of the invention;

FIG. 11 shows an interface for selecting domains when creating or modifying a therapy plan in accordance with embodiments of the invention;

FIG. 12 shows an interface for annotating programs when creating or modifying a therapy plan in accordance with embodiments of the invention;

FIG. 13 shows an interface for adding a behavior intervention plan (BIP) in accordance with embodiments of the invention;

FIG. 14 shows a screenshot showing information related to a behavior intervention plan (BIP) in accordance with embodiments of the invention;

FIG. 15 shows a screenshot showing a client dashboard which is associated with a particular subject in accordance with embodiments of the invention;

FIG. 16 shows a screenshot that includes a display of a staff member's name and title, a list of notes, and a list of sessions, in which each session is associated with a particular client (or subject), a particular time in, a particular time out, and a particular status, as well as a user interface element button which can be activated to view notes from a particular session in accordance with embodiments of the invention;

FIG. 17 shows a login screen of a mobile app running on a portable electronic device in accordance with embodiments of the invention;

FIG. 18 shows a screenshot that includes a “Programs” tab which is associated with a particular client or subject in accordance with embodiments of the invention;

FIG. 19 shows an interface for entering criteria used to “probe” a target in accordance with embodiments of the invention;

FIG. 20 shows a “Trials” display with information associated with a particular subject in accordance with embodiments of the invention;

FIG. 21a shows an alert dialog box which appears if the system and method determine, based on the outcome of probes or trials, that a skill associated with a target is known by a subject;

FIG. 21b shows a representative embodiment of the invention including an alert dialog box which appears if the system and method determine, based on the outcome of probes or trials, that a skill associated with a target is not known;

FIG. 22 shows screenshot of a “Trials” display with information associated with a particular subject (a.k.a., “client”), where this display includes trial data in accordance with embodiments of the invention;

FIG. 23 shows a screenshot of a “Trials” display with information associated with a particular subject in accordance with embodiments of the invention;

FIG. 24 shows a screenshot of a “Behaviors” display which is used in the creation or implementation of a behavior intervention plan (BIP) in accordance with embodiments of the invention;

FIG. 25 shows a screenshot of a “Skills” display which is used in functional skills training for a subject in accordance with embodiments of the invention;

FIG. 26 shows a screenshot of a “Maintenance” display with information associated with a particular subject in accordance with embodiments of the invention;

FIG. 27 shows a screenshot of a “Daily Session Notes” display with information associated with a particular subject in accordance with embodiments of the invention; and

FIG. 28 is a schematic diagram illustrating an overall view of communication devices, computing devices, and mediums for implementing embodiments of the invention.

### DETAILED DESCRIPTION OF THE INVENTION

The present invention has utility as a system and method for automatically conducting tasks in support of autism therapy sessions for a human subject. Computerized methods are provided for automatically conducting tasks in support of autism therapy. These computerized methods may include creating therapy plans, or modifying therapy plans, by using a mathematical model to process data collected from assessments or from therapy sessions. Applying embodiments of the mathematical model to therapy session data may be used to generate a new or a revised therapy plan for the subject. In some inventive embodiments, there may be multiple iterations of the process of collecting therapy session data, generating a new or a revised therapy plan from the data, and applying this therapy plan in subsequent autism therapy.

In inventive embodiments, the creation of an autism therapy plan includes the identification of a skill or skills that the subject is required to master, and the creation of a program for each of these skills. Each of these programs can, in turn, be created by creating or identifying: goals, targets, instructions, and mastery criteria. Non-limiting examples of therapy plans which may be created in this way include a behavior therapy plan (BTP), a behavior intervention plan (BIP), or a plan of service (currently working programs).

Method embodiments of the invention in some embodiments may include the steps of: conducting a subject assessment to generate assessment results relating to degrees of autism of the human subject; documenting the assessment results electronically; creating an autism therapy plan by inputting the assessment results into a mathematical model; implementing the autism therapy plan by conducting therapy programs; monitoring therapy program results, and creating a revised autism therapy plan which is generated using the therapy program results as an input.

The following definitions apply to terms that are used throughout the invention disclosure.

As used herein, a “program” is defined as a subcomponent of an autism therapy plan, and is associated with a single skill that is intended to be taught to the subject.

As used herein, “goal” is defined herein as a subcomponent of a program, and indicates the desired level of proficiency related to a skill.

As used herein, “target” is defined as a subcomponent of a program. The goal is not uniquely tied to specific targets so both goals and targets are related to programs and are at the same level. However, in a specific embodiment, a program may have a hierarchy that deconstructs to a set of goals, which in turn deconstruct to a set of targets.

As used herein, “instructions” constitute directions meant to be followed by a therapy provider when carrying out the therapy.

As used herein, “mastery criteria” constitute criteria used to assess and indicate whether or not a target (or sub skill) goal has been achieved by the subject.

Once an autism therapy plan is created, therapy sessions can be conducted. During these sessions, a therapist and a subject may attempt to complete trials on targets to complete programs which are part of the autism therapy plan.

During therapy or an assessment, a probe may be used for a new target to assess proficiency before continuing with additional trials. As used herein, a “probe” is a test or a mini-assessment which determines the proficiency level of a subject in regard to a specific skill before continuing with additional trials. A probe is a type of trial but refers to the first time the target is initiated. The target is ‘probed’ to understand if the client already has command of the particular skill. As such, the available responses are ‘known’ and ‘unknown’. If ‘known’ the skill moves immediately to maintenance as a mastered target. If not, further trials are conducted on the skill using the more elaborate options for capturing the response.

During a therapy session, a target may be selected from a program. A computer system can determine whether a selected target has been started already. The computer system may include logic to automatically determine if a selected target is in probe mode—that is, whether certain “probe criteria” have or have not been satisfied, and/or whether the subject has started to work on a target.

If a subject has not yet started to work on a target, an interface included in the computer system may be used to enter probe criteria. Also, data may be entered into the computer system which indicates whether each probe is “known” or “unknown”, meaning whether the subject has been determined to possess a skill associated with that probe (i.e., a skill that the probe in question is designed to test for). Other data in the computer system may designate probes as being part of a list, where each probe has a unique number or other label associated with it.

Additional logic in the computer system may determine whether a probed target requires further data collection. If probe criteria have not been satisfied, the probed target is placed in an “acquisition” list, indicating that the skill associated with the probed target still needs to be acquired by the subject. If probe criteria have been satisfied, however, the probed target is moved to a “maintenance” list, which indicates that a skill has been mastered or acquired and that future therapy will be directed to maintaining the skill over time.

Creation of a new or a revised autism therapy plan may include calculating new programs, new targets, or a new combination thereof by inputting data into a mathematical model. This data may include information collected from subject assessments or information collected during autism therapy sessions, or both. When revising a plan, this data may also include information from acquisition lists and maintenance lists which were created during earlier therapy sessions.

In some inventive embodiments, documenting results of the subject assessment includes: navigating to a portal using a web browser; selecting a client from a client list within the portal; selecting a user-interface element which is associated with the client; opening a note element which is associated with the user interface element, and populating the note element with information from an existing note element.

In other inventive embodiments, creating an autism therapy plan includes one or more of: identifying a number of required skills; creating a program for each of the required skills; identifying program goals; identifying program targets; identifying program instructions; and identifying program and mastery criteria.

In other inventive embodiments, conducting a subject assessment includes: initiating a functional assessment; selecting an assessment; conducting the selected assessment; and completing the functional assessment.

In other inventive embodiments, a verbal behavior milestones assessment and placement program (VB-MAPP), or an assessment of basic language and learning skills (ABLLS) are used as a way of organizing our standard program library to ease development of the BTP.

In other inventive embodiments, implementing the autism therapy plan includes: setting up data sheets; initiating progress notes by listing programs and targets; selecting certain selected programs to work on; and completing the certain selected programs.

In other inventive embodiments, completing the certain selected programs includes: running trials on new targets; and completing maintenance on previously completed targets.

In other inventive embodiments, completing certain selected programs includes: selecting a target from certain selected programs; conducting probes or trials; and determining whether a number of consecutive sessions have been completed.

In other inventive embodiments, completing the certain selected programs includes: selecting a target from a certain selected program; identifying probe criteria; conducting probes; and determining whether a number of consecutive sessions have been completed.

In some inventive methods, the method additionally includes: providing a notification prompt to take a break; and recording a description of how a program was completed.

In other inventive methods, the method additionally includes: completing a behavior intervention plan; completing progress notes with automatically recorded target percentages and problem behaviors; documenting subject behaviors; and closing out the autism therapy session.

In other inventive embodiments, monitoring the autism therapy plan includes: determining behaviors for a behavior intervention plan.

In other inventive embodiments, creating a revised autism therapy plan includes: selecting programs, targets, or a combination thereof from the autism therapy plan; determining whether or not at least one program, at least one target, or a combination thereof has been mastered; electronically documenting mastered programs or mastered targets in a maintenance list; electronically documenting non-mastered programs or non-mastered targets in an acquisition list; calculating new programs, new targets, or a new combination thereof by using the maintenance list and the acquisition list as inputs to a mathematical model; and integrating the new programs, new targets, or new combination into a revised autism therapy plan.

In autism therapy, methods of using a computing device to on-board and assign clients and staff in support of autism services may include the steps of: registering through the use of a web portal; verifying payment information through the web portal; creating an agency database; providing a login link to an administrator; accessing the agency database through the login link; adding autism clients and staff to the agency database; assigning staff to autism clients; and creating an autism behavior therapy plan.

In some inventive methods, the method additionally includes: viewing a client's autism behavior therapy plan.

In some methods, the method additionally includes: emailing login credentials.

In autism therapy, methods of using a mobile device to initiate autism therapy sessions may include the steps of: logging in; selecting a client; selecting a program for use in autism therapy sessions with the client; applying filters to the program; viewing the program; entering a planned session duration; and starting a session.

In particular embodiments of the method, the method additionally includes: applying target filters after viewing the program.

In autism therapy, inventive methods of using a mobile device to conduct trials in autism therapy sessions may include the steps of: selecting a program; selecting a target; using logic to automatically determine if the selected target is in probe mode; and if the logic determines that the selected target is in probe mode, entering or populating probe details, and recording probe details using electronic documentation; and alternatively, if the logic determines that the selected target is not in probe mode, recording trials using electronic documentation. This method may be performed for direct financial remuneration from the subject or from a third-party payor illustratively including an insurance company or a governmentally funded social service, or medical provider.

An inventive system is provided that includes a computing device operating software adapted to automatically conduct tasks in support of autism therapy sessions for a human subject, the tasks including: conducting a subject assessment to generate assessment results relating to degrees of autism of the human subject; documenting the assessment results electronically; creating an autism therapy plan by inputting the assessment results into a mathematical model; implementing the autism therapy plan by conducting therapy programs; monitoring therapy program results, and creating a revised autism therapy plan which is generated using the therapy program results as an input. A display and user interface are provided with the computing device. The system may be supplied to therapists and supported for direct financial remuneration from the subject or from a third-party payor such as an insurance company or a governmentally funded social service, or medical provider.

The inventive embodiments disclosed in the drawings are for illustrative purposes only, and are not intended to be limiting.

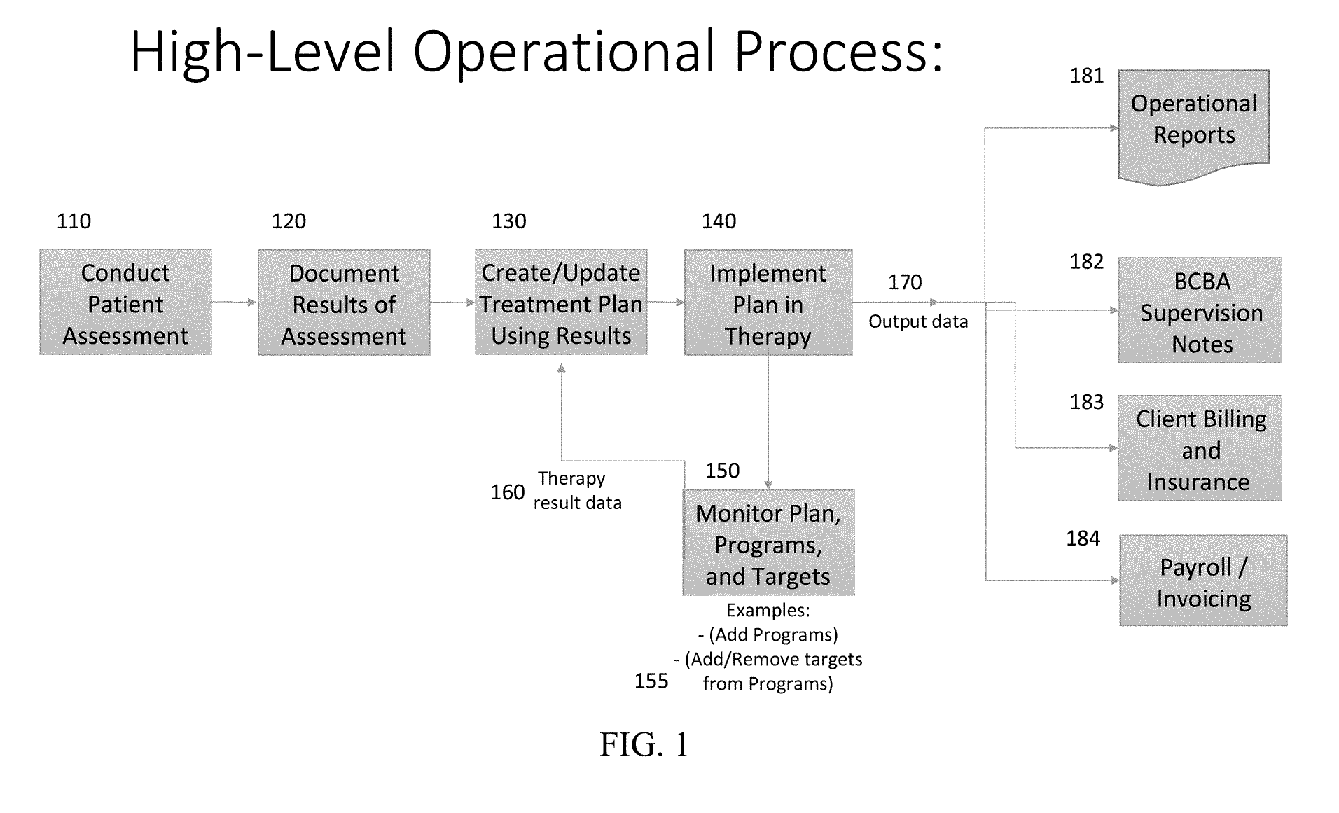

Referring now to the figures, FIG. 1 is a flow diagram of a high-level overview of a process for using a computing device to automatically conduct tasks in support of autism therapy sessions for a human subject. A subject assessment is conducted at step 110. The results of this subject assessment are documented at step 120. A therapy plan is then created at step 130, using the results of the subject assessment as an input. The therapy plan, or part of the therapy plan, is then implemented in the context of autism therapy at step 140. The implementation of the therapy plan, including implementation of the programs and targets of the therapy plan, is monitored at step 150. A subcomponent of this may be adding programs to the plan, or adding/removing targets to the plan, at step 155. Therapy result data which is monitored during the session is then used as an input, at step 160, to update the existing therapy plan, thus returning to step 130. The implementing, monitoring, and updating steps can be undergone multiple times, resulting in a new, updated therapy plan at every iteration. Output data 170 resulting from implementing the therapy plan may be used as input in creating operational reports 181 or supervision notes 182. Output data can additionally be used for client billing and insurance claims 183, and can provide details needed for payroll or invoicing purposes 184.

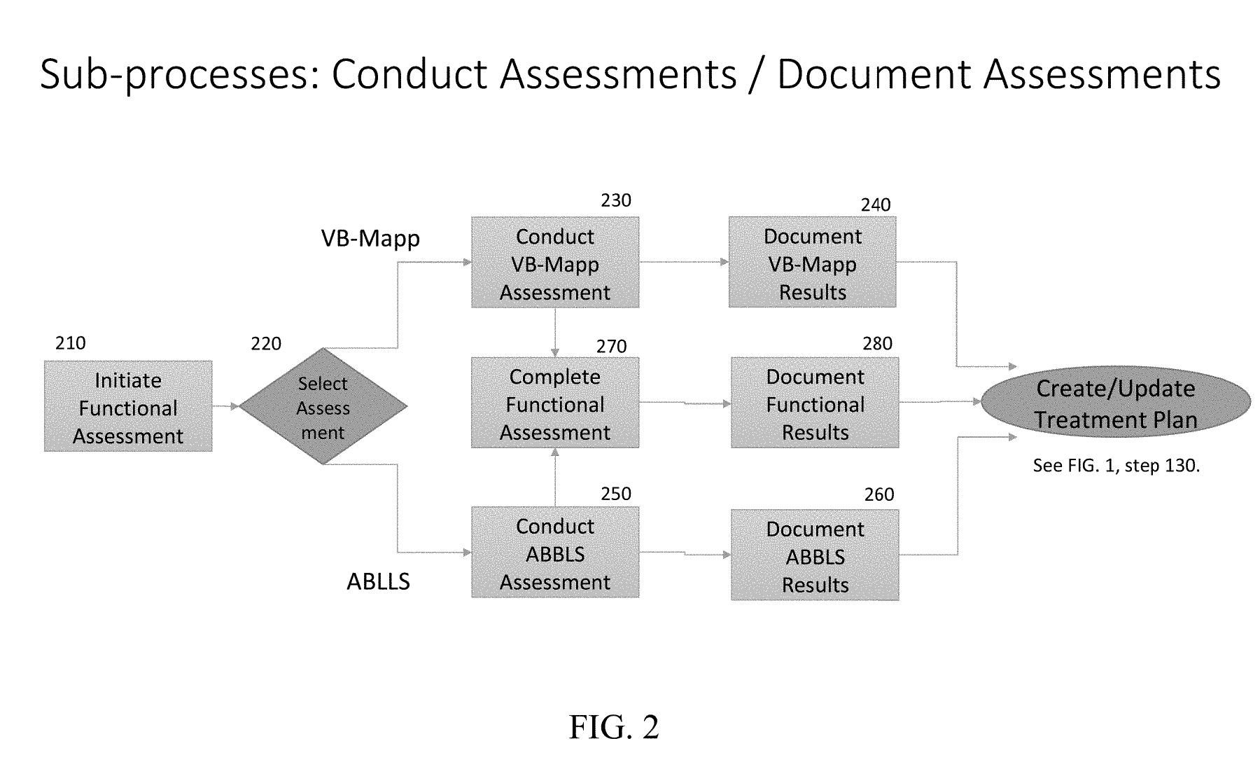

FIG. 2 is a flow diagram representing possible sub-processes of the steps of “Conduct Subject Assessment” from FIG. 1, step 110 and “Document Results of Assessment” from FIG. 1, step 120. A functional assessment is initiated at step 210. A type of assessment is selected at step 220. The selected assessment might be a VB-MAPP assessment; if so, the VB-MAPP assessment is conducted at step 230 and its results are documented at step 240. The selected assessment might alternatively be an ABBLS assessment; if so, the ABBLS assessment is conducted at step 250 and its results are documented at step 260. The functional assessment is completed at step 270 and the results of the functional assessment are documented at step 280. The results of the VB-MAPP, ABBLS, and functional assessments are used to create or update the therapy plan, as takes place in FIG. 1, step 130.

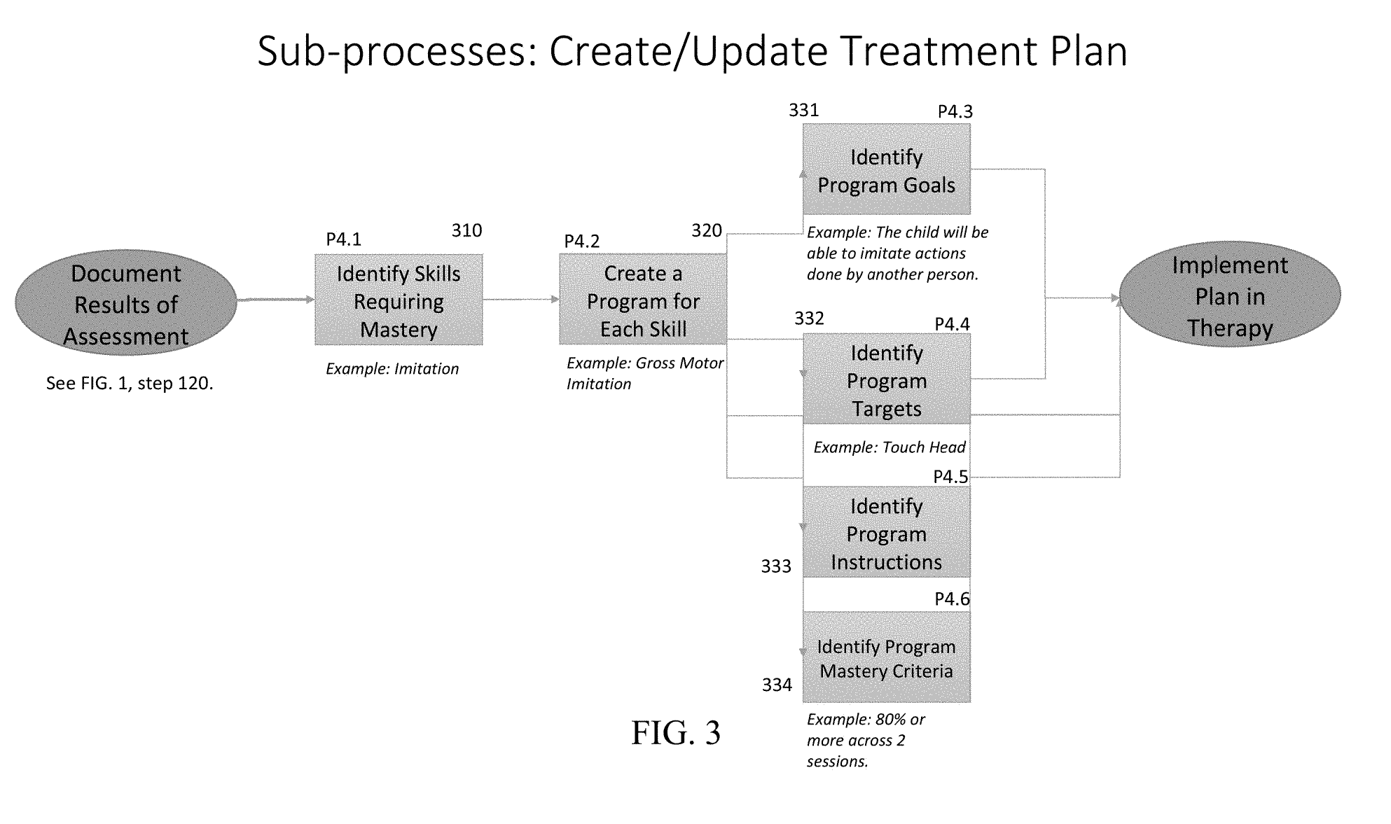

FIG. 3 is a flow diagram representing possible sub-processes of the steps of “Create/Update Therapy Plan Using Results” from FIG. 1, step 160. In some inventive embodiments, the steps herein can be carried out by a behavior consultant (BC) or board-certified behavior analyst (BCBA), whereas therapists conduct therapy sessions on the direction of the BC or BCBA. An initial step is to identify skills requiring mastery at step 310; this may be done by using scores from assessment results. A next step is to create a program for each skill at step 320. This may be done by identifying program goals, program targets, program instructions, and program mastery criteria at steps 331, 332, 333, and 334. These program goals, program targets, program instructions, and program mastery criteria are then incorporated into therapy plans. Examples of such therapy plans might include a behavior treatment plan (BTP), or a behavior intervention plan (BIP), or a plan of service which relates to programs that are currently being worked on by a subject

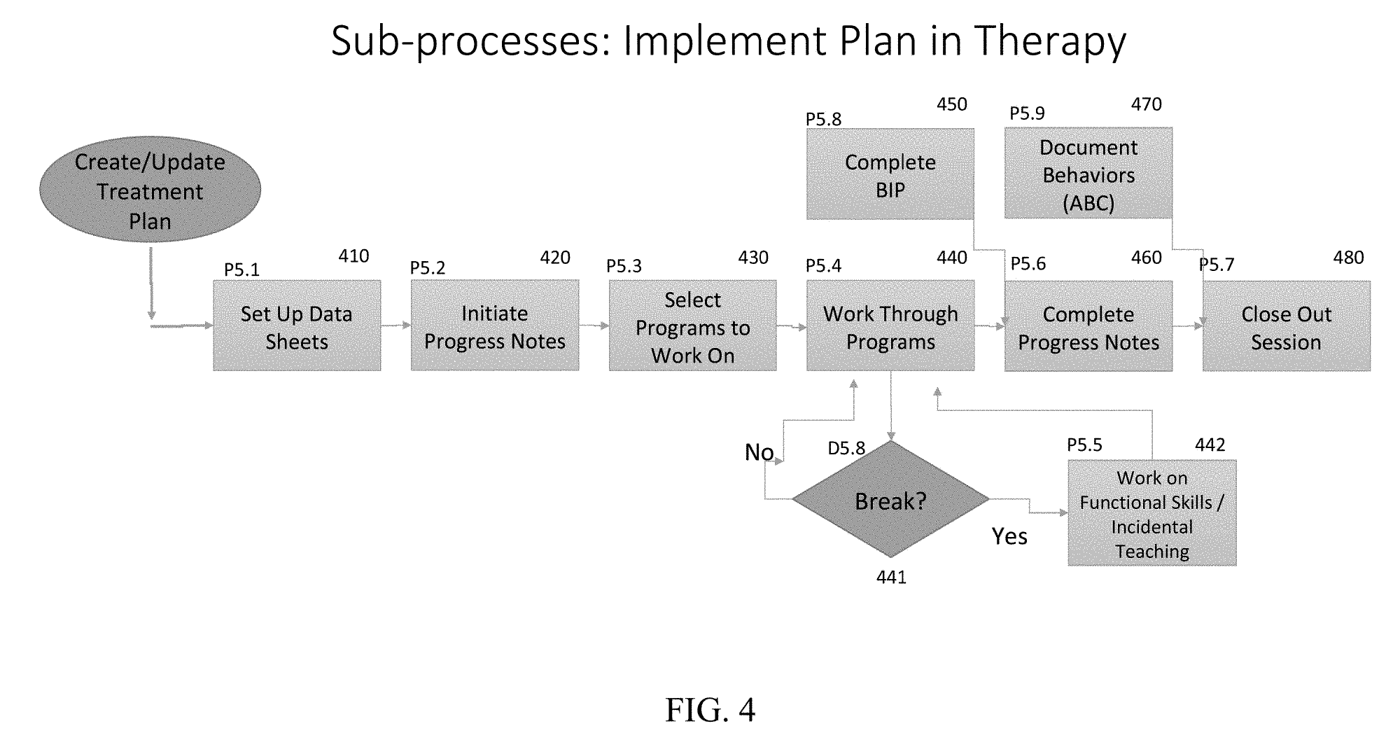

FIG. 4 is a flow diagram representing possible sub-processes of the steps of “implement plan in therapy” from FIG. 1, step 140. In some inventive embodiments, the steps herein can be carried out by a behavior technician conducting the therapy session under the guidance of the BC or BCBA.

An initial step is to set up data sheets for each program at step 410; this may include adding titles and tasks for each data sheet. A next step is to initiate progress notes, which may include lists of programs and targets, at step 420. A next step is to select which programs to work on, and the order in which to work on them, at step 430. The subject, through therapy, will then work through a program at step 440. At some point, the subject may be allowed to take a break (e.g., every 15 minutes). In the flow chart, the decision point regarding whether or not to take a break occurs at step 441. If a break is taken, the opportunity may be used to work on functional skills or incidental teaching, and to record related data, at step 442. If a break is not taken, therapy continues, the process returns to step 430, and another program to work on can be selected. The end of a therapy session could include the completion of a behavior intervention plan (BIP) at step 450, the completion of progress notes at step 460, documenting behaviors at step 470, and closing out a session at step 480.

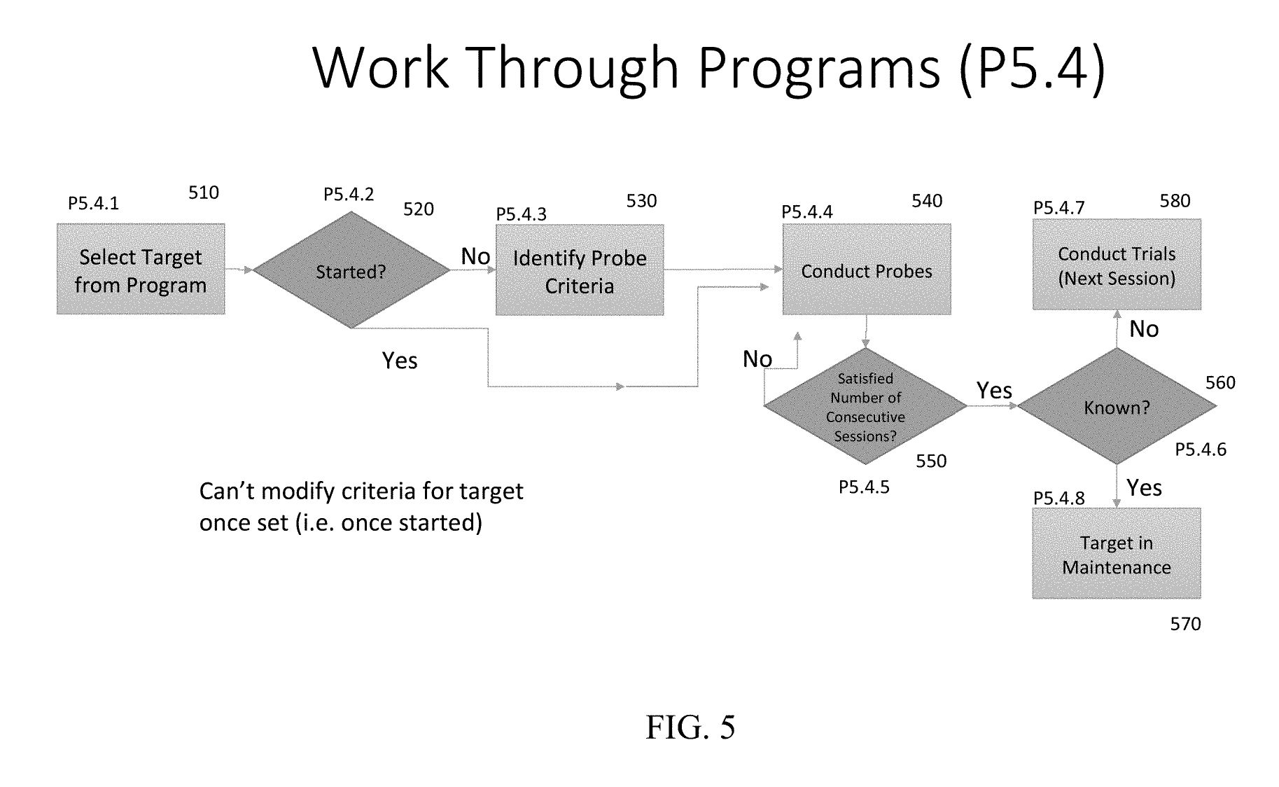

FIG. 5 is a flow diagram representing possible sub-processes of the step of “Work Through Programs” from FIG. 4, step 440. An initial step is to select a target from a program at step 510. At step 520, a determination is made whether this selected target has or has not been started. If the target has not been started, probe criteria are identified at step 530, after which probes are conducted at step 540. If the target has been started, the step 530 of identifying probe criteria is skipped and the process goes straight to step 540, where probes are conducted. Following this, a determination is made at step 550 whether the subject has satisfactorily completed a number of consecutive sessions; if not, the process returns to step 540 and an additional probe or probes are conducted. If yes, a determination is made at step 550 whether the subject has acquired a skill; that is, whether a skill is “known” or not. If yes, the target is placed in a maintenance list at step 570. If not, a designation is made to conduct trials on the target at a next therapy session at step 580.

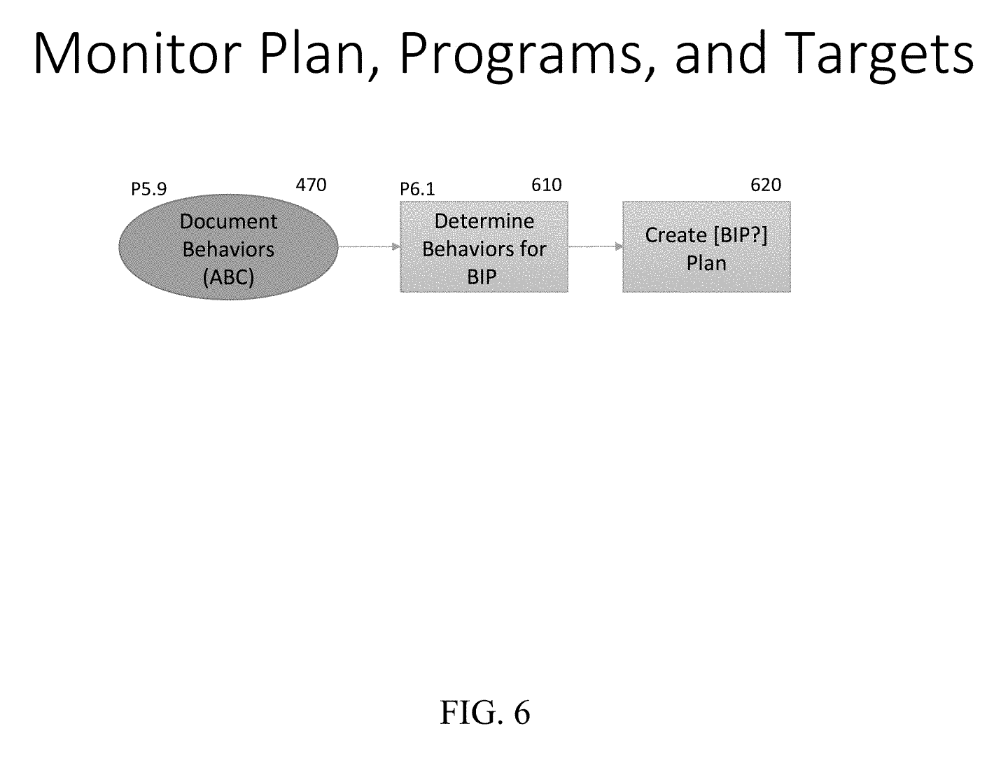

FIG. 6 is a flow diagram representing possible sub-processes of the step of “Monitor Plan” from FIG. 1, step 150. An initial step is to document subject behaviors associated with a sequence of “ABC” (Antecedent, Behavior, Consequence), which is a component of ABA therapy; this sequence includes observing subject behavior, as well as an event preceding the behavior (“antecedent”) and an event following the behavior “consequence”). Following this, behaviors are determined for use in a behavior intervention plan (BIP) at step 610. Following this, a BIP is created at step 620.

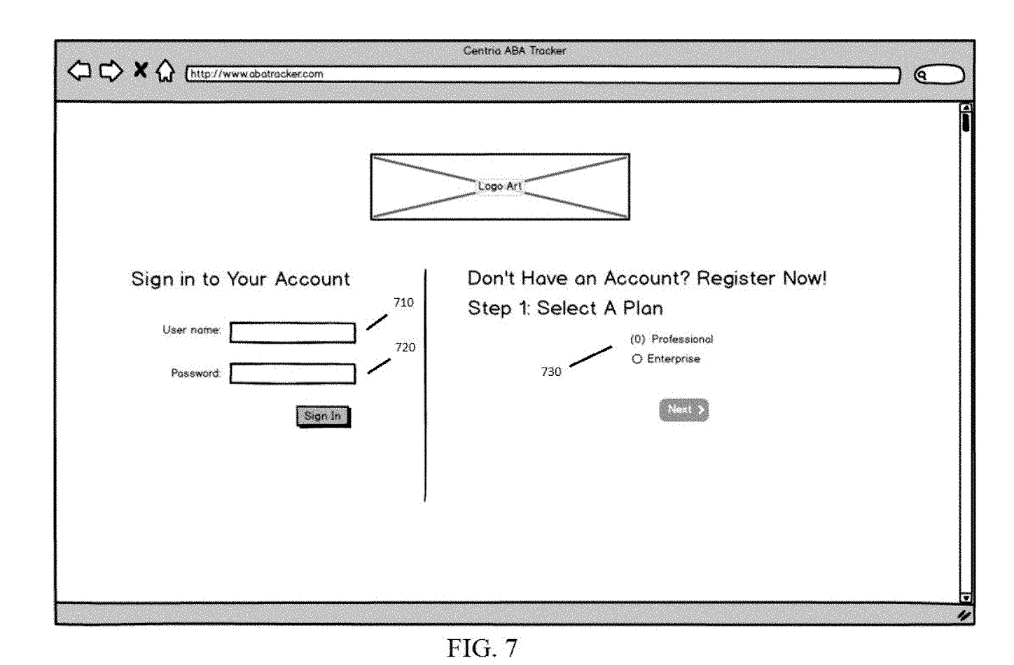

FIG. 7 shows a representative embodiment of the invention including a login screen for a web portal. The login screen includes fields for entering a username 710 and password 720, and an account type selector 730.

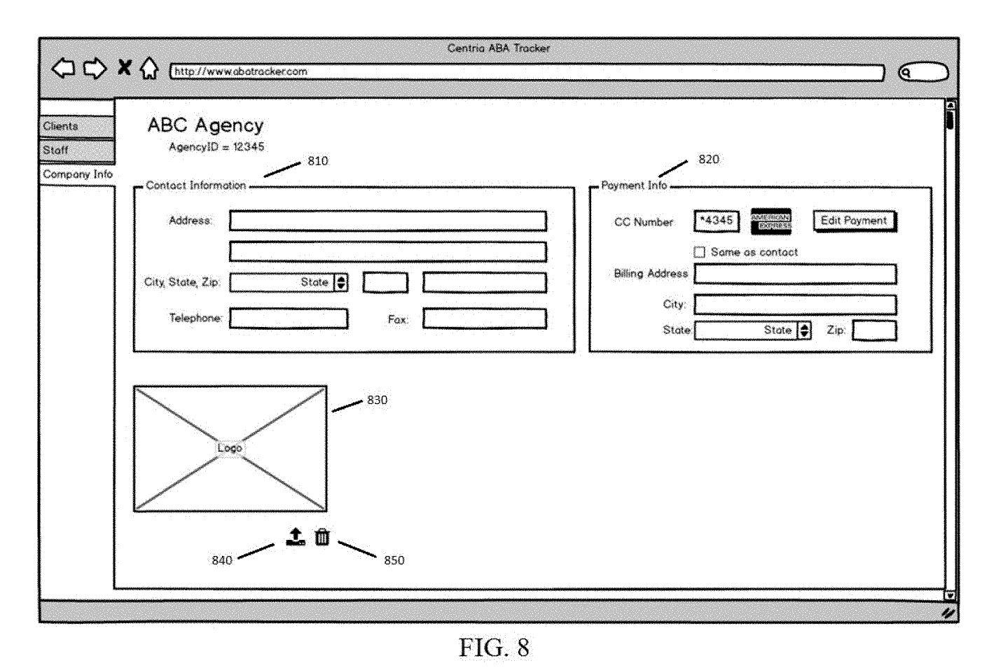

FIG. 8 shows a representative embodiment of the invention including a user account information screen, containing information related to a user's web portal account (an agency might be one such user). The account information screen includes areas for entering a user's contact information 810, payment information 820, and an area for showing a user's logo 830. Features of such an account information screen could include clickable icons for uploading a user's logo 840, or for deleting a user's logo 850.

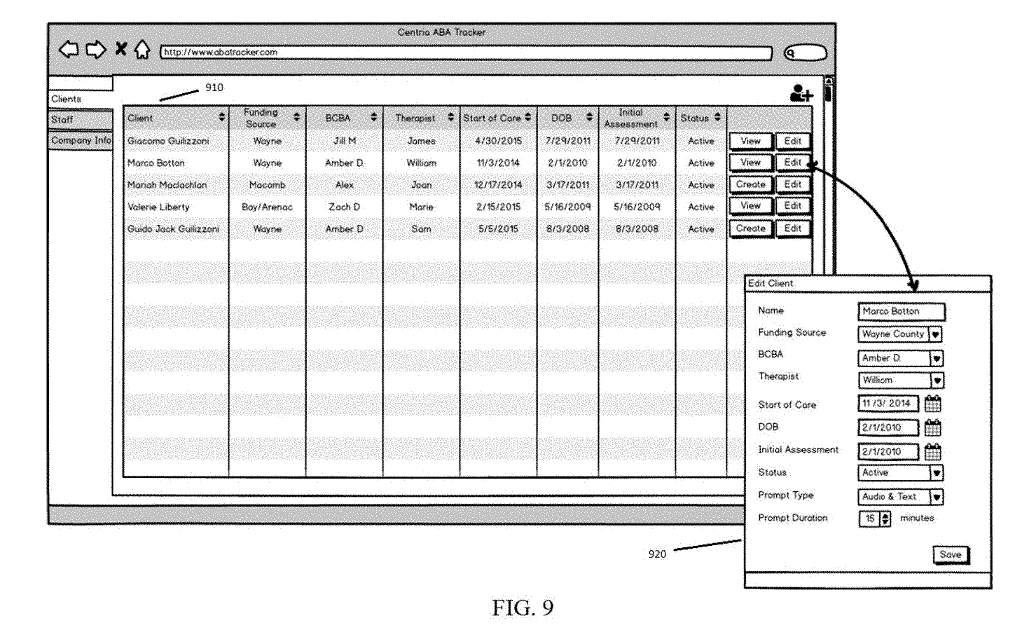

FIG. 9 shows a representative embodiment of the invention including a client information screen. Each client listed in the information grid 910 represents information associated with an autism subject. Each line contains information associated with a different autism subject. An “Edit” button on each line causes a sub-screen 920 to manifest, which can be used to edit client information associated with the autism subject listed on that line.

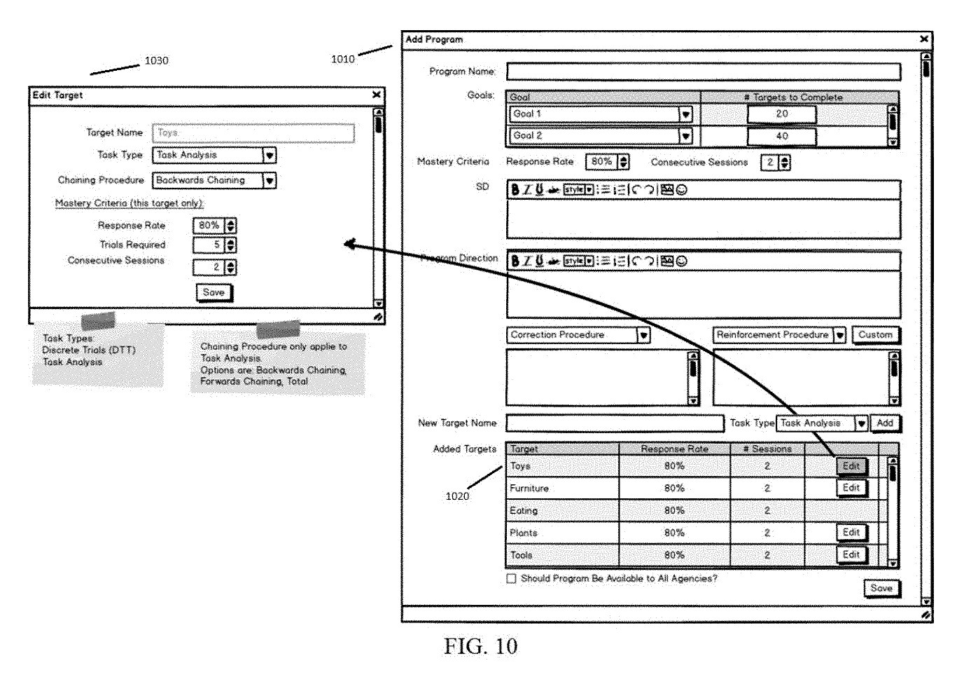

FIG. 10 shows a representative embodiment of the invention including an interface for managing therapy programs. An “Add Program” screen 1010 is used to input information associated with a program, including program name, goals, mastery criteria, SD, program direction, correction procedure, names of targets, and task type. This screen includes a grid 1020 which contains information about targets, such as name, response rate, and number of sessions. Information about each target can be edited through the use of an “Edit” button, which causes a sub-screen 1030 to manifest which can be used to edit information associated with that target.

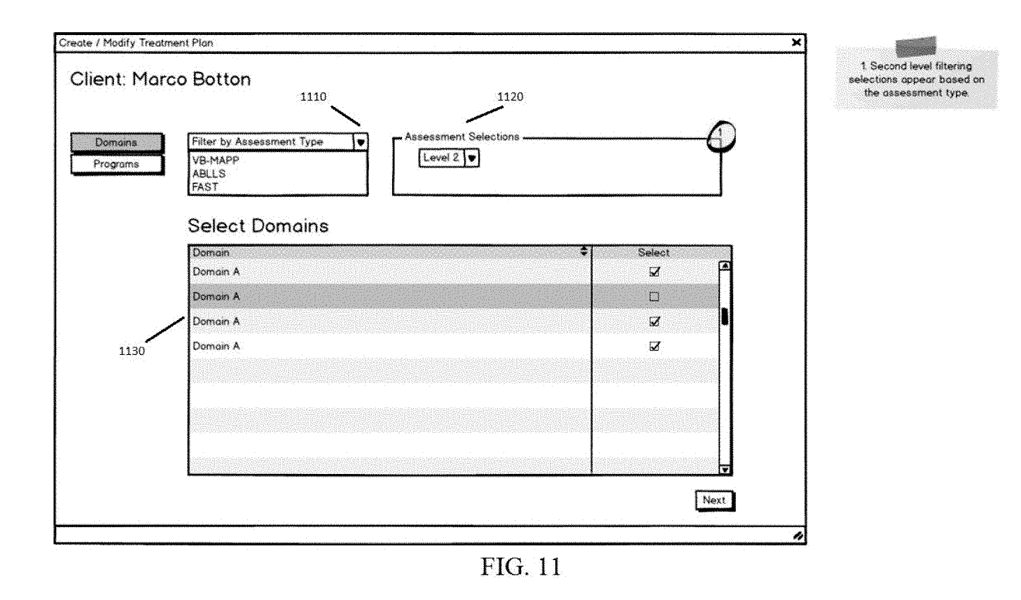

FIG. 11 shows a representative embodiment of the invention including an interface for selecting domains when creating or modifying a therapy plan. A drop-down selector menu 1110 is used to select an assessment type which can be used for filtering; examples of assessment types could include verbal behavior milestones assessment and placement program (VB-MAPP), assessment of basic language and learning skills (ABLLS), and functional analysis screening tool (FAST). Another drop-down selector menu 1120 can then be used to select an assessment, and a domain selector 1130 can be used to select domains (in this context, “domains” means assessment categories). In some inventive embodiments, second-level filtering selections can appear at 1120 or 1130 based on the assessment type selected at 1110.

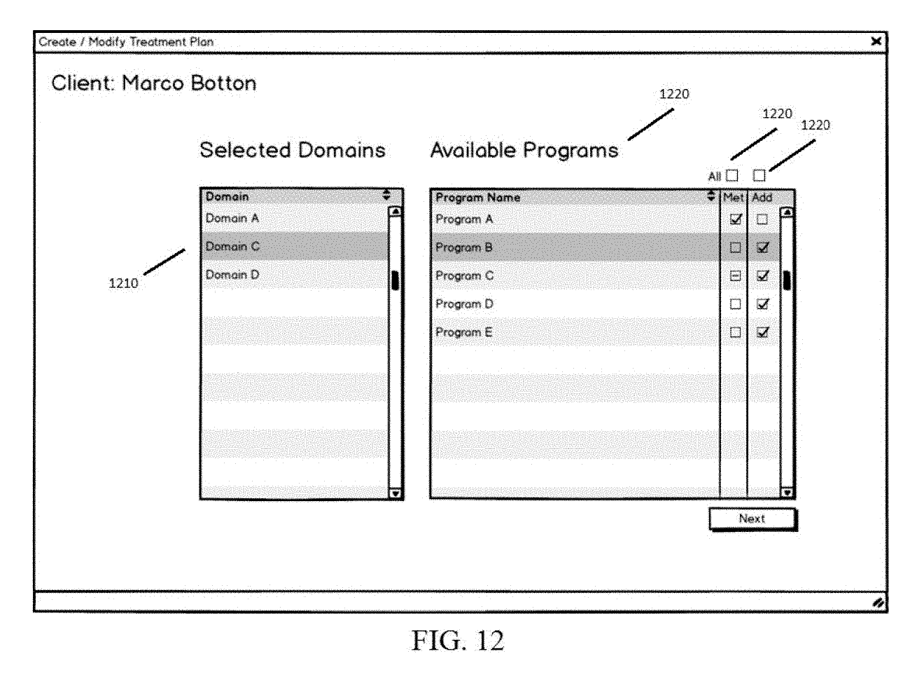

FIG. 12 shows a representative embodiment of the invention including an interface for annotating programs when creating or modifying a therapy plan. A list of domains which were selected using a previous domain-selecting interface (such as the interface in FIG. 11) appears at 1210. A grid, containing available programs which are mapped to selected domains, appears at 1220. This grid includes program names which are associated with a “Met” column and an “Add” column. The interface allows a user to mark a box in the “Met” column, indicating that a program should be placed in a maintenance list; or to mark a box in the “Add” column, indicating that a program should be added to an acquisition list.

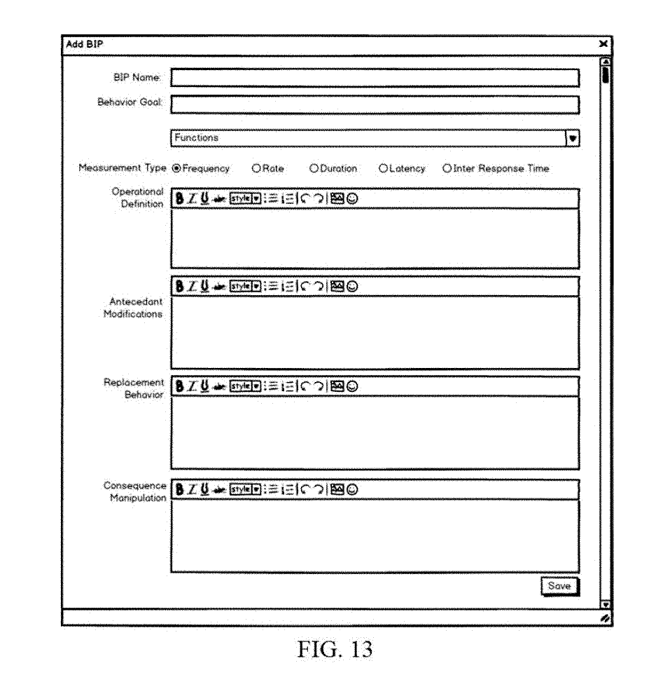

FIG. 13 shows a representative embodiment of the invention including an interface for adding a behavior intervention plan (BIP). Using this interface, a BIP is created by inputting information such as the BIP name, behavior goals, functions, measurement type, operational definitions, antecedent modifications, replacement behavior, and consequence manipulation.

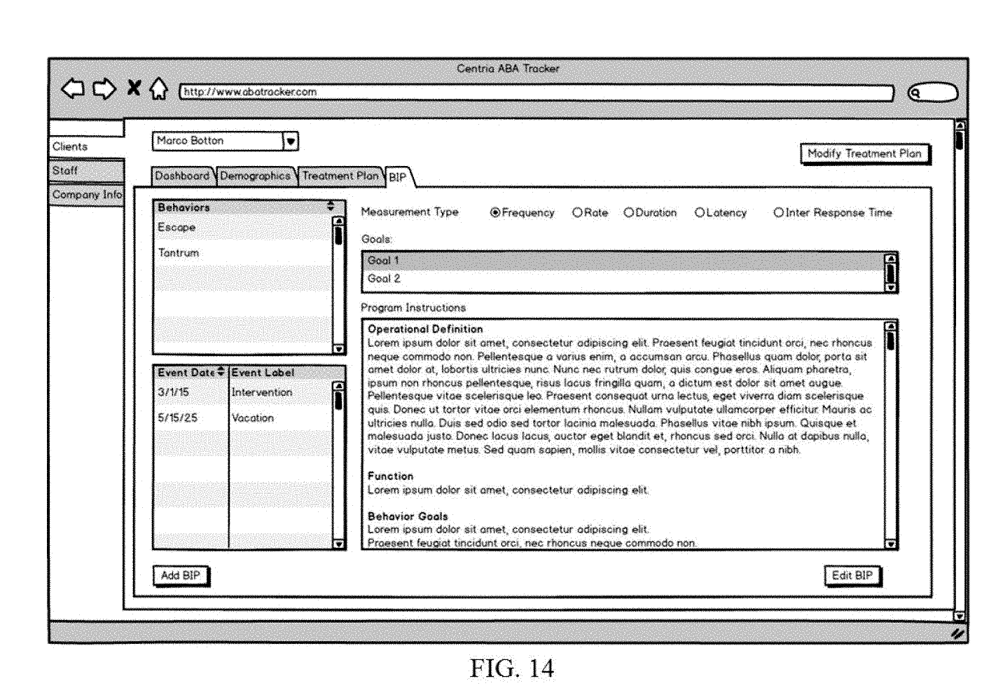

FIG. 14 shows a representative embodiment of the invention including a display showing information related to a behavior intervention plan (BIP). The information may include behaviors, events and event information, measurement type, goals, and program instructions associated with the goals. In some inventive embodiments, the information is received by being input into the interface in FIG. 13.

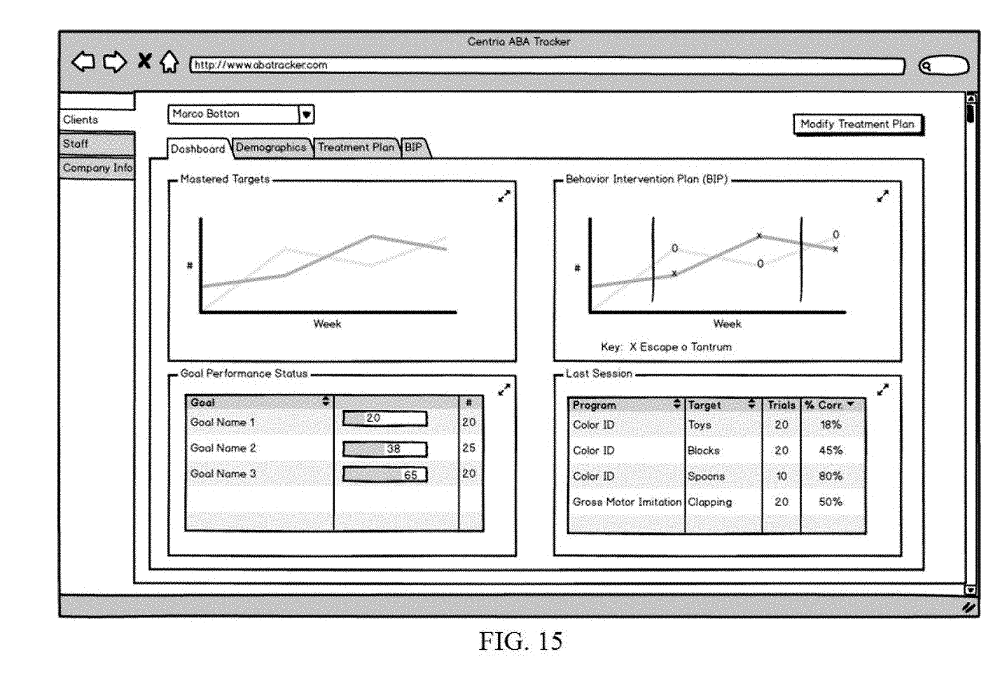

FIG. 15 shows a representative embodiment of the invention including a display of a client dashboard which is associated with a particular subject, which includes numerical and visual information relating to a client's mastered targets, behavior intervention plan (BIP), goals performance status, and information relating to a client's previous or last session including programs, targets, number of trials, and percentage correct.

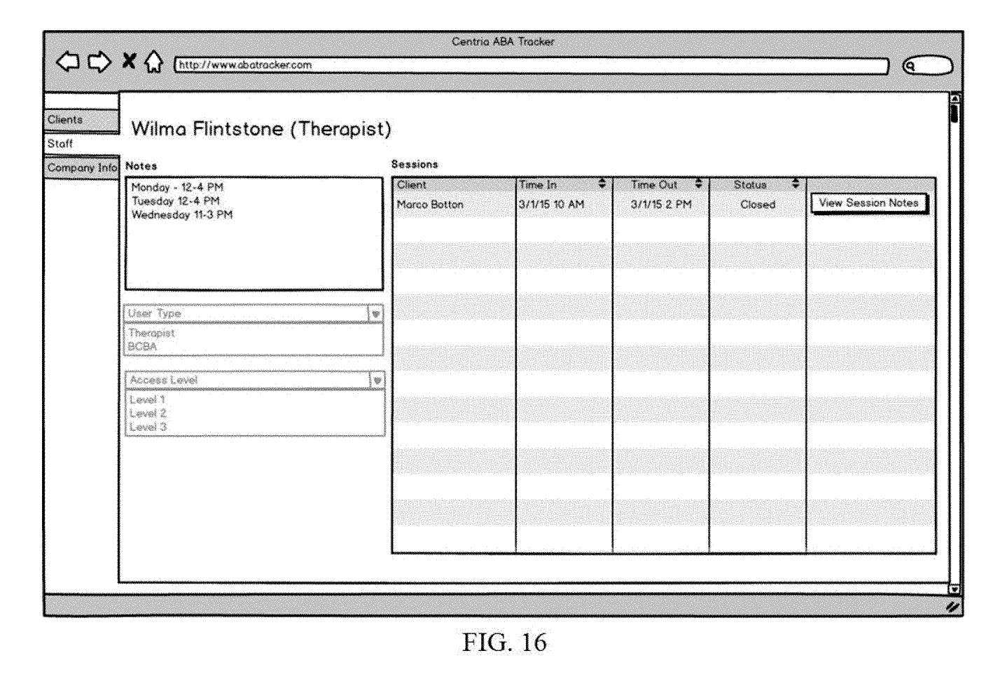

FIG. 16 shows a representative embodiment of the invention including a display of a staff member's name and title, a list of notes, and a list of sessions, in which each session is associated with a particular client (or subject), a particular time in, a particular time out, and a particular status. The display also includes a user interface element button which can be activated to view notes from a particular session.

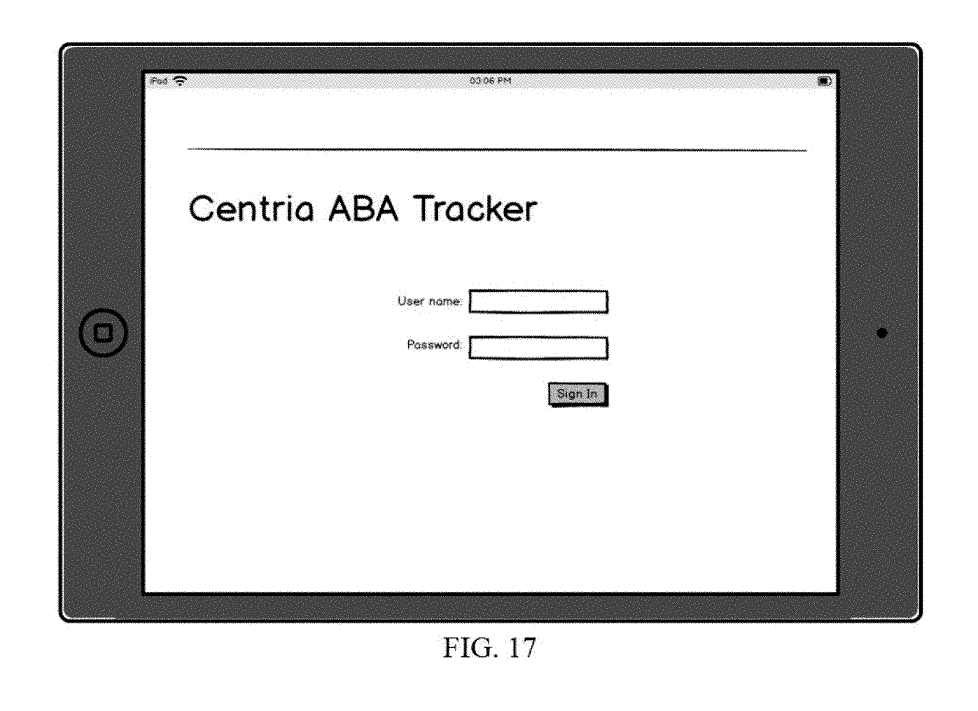

FIG. 17 shows a representative embodiment of the invention including a login screen of a mobile app running on a portable electronic device. The login screen includes fields for entering a user name and a password.

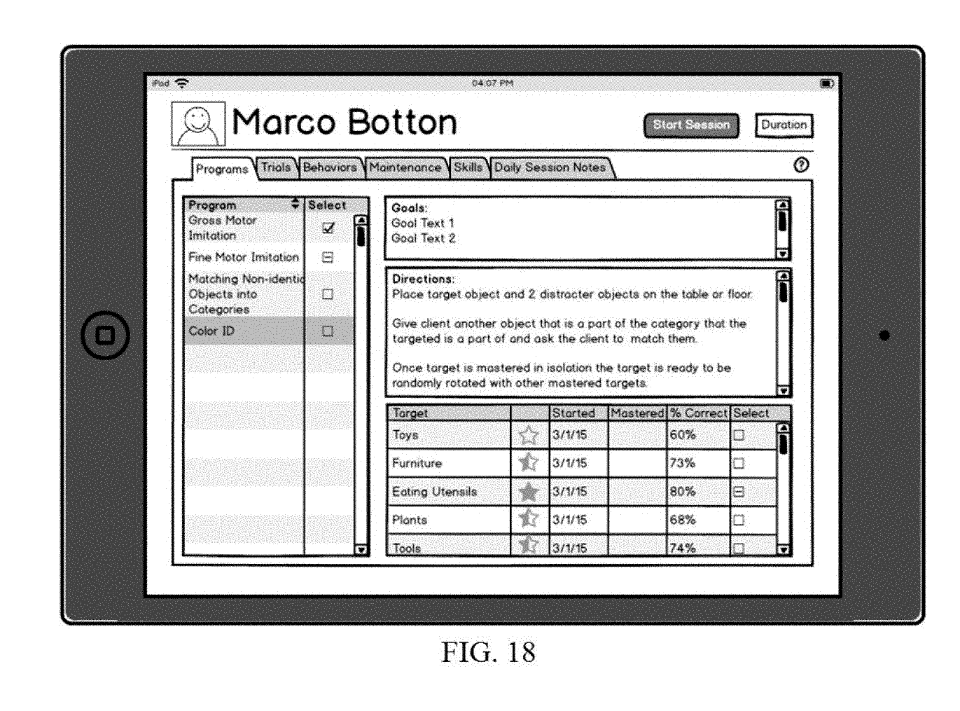

FIG. 18 shows a representative embodiment of the invention including a “Programs” tab which is associated with a particular client or subject. This Programs tab includes a list of programs, a list of goals, a field containing directions, and a grid containing information relating to targets such as names, date started, and percentage correct.

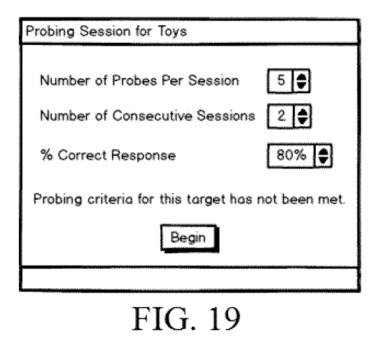

FIG. 19 shows a representative embodiment of the invention including an interface for entering criteria used to “probe” a target, i.e., to test whether a subject knows or does not know a particular skill. The interface includes a title bar which displays the name of the skill to be probed, and also includes entry boxes for selecting the number of probes per session, number of consecutive sessions, and a minimum percentage correct threshold which must be met in order to meet the criteria, which in this context are known as “probe” criteria.

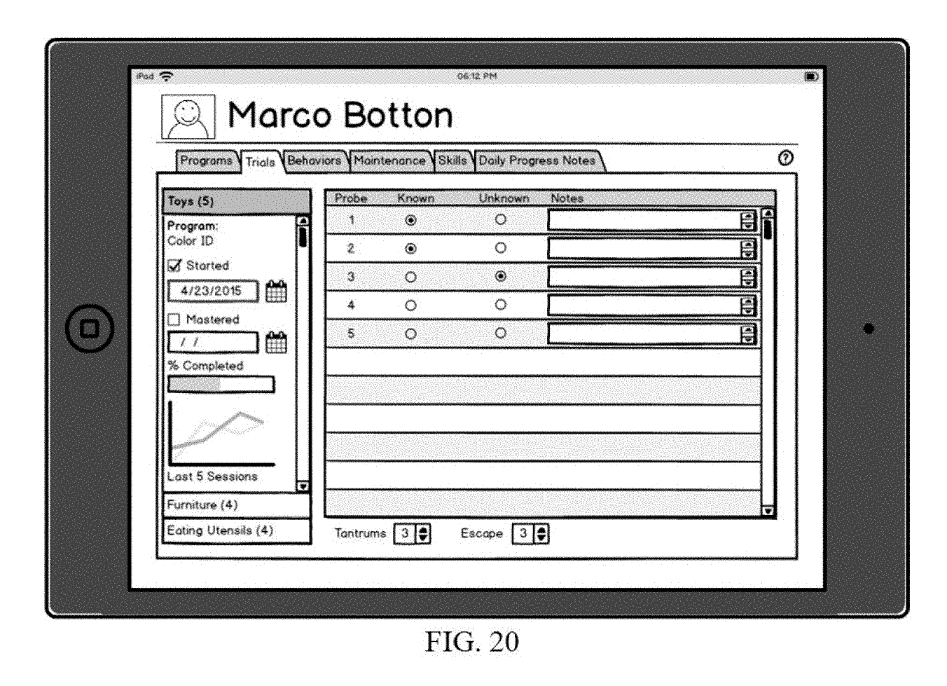

FIG. 20 shows a representative embodiment of the invention including a “Trials” display with information associated with a particular subject (a.k.a., “client”), where this display includes probe data. The information on this display also includes a name of a program being worked on (“Color ID”, in this case), the target being worked on (“Toys”, meaning in this case, correctly identifying what color a toy is), and a start date. This display also includes a grid which lists probes that have or will be conducted, along with a selector which indicates whether each probe showed a skill to be known or unknown by the subject, and a data entry field for notes.

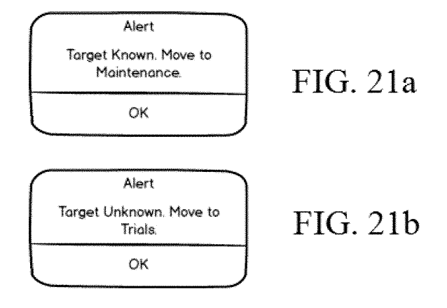

FIG. 21a shows a representative embodiment of the invention including an alert dialog box which appears if the system and method determine, based on the outcome of probes or trials, that a skill associated with a target is known by a subject. FIG. 21b shows a representative embodiment of the invention including an alert dialog box which appears if the system and method determine, based on the outcome of probes or trials, that a skill associated with a target is not known (or unknown) by a subject.

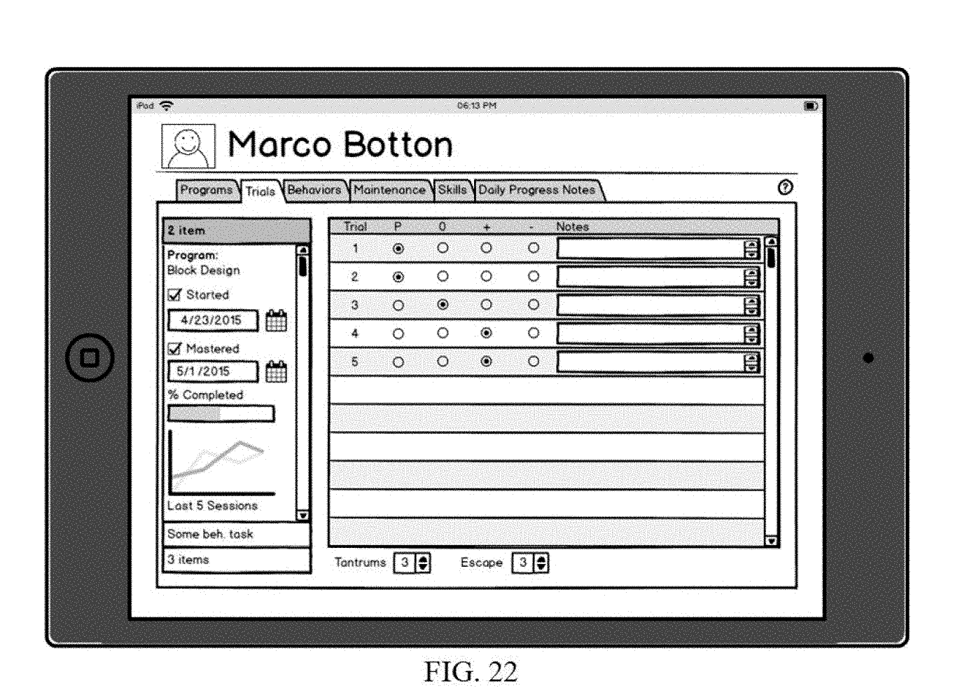

FIG. 22 shows a representative embodiment of the invention including a “Trials” display with information associated with a particular subject (a.k.a., “client”), where this display includes trial data. The information on this display also includes a name of a program being worked on (“Block Design”, in this case), a start date, and a date showing when a particular skill was mastered. This display also includes a grid which lists trials that have been conducted, along with a selector which can be used to select a status indicator for each trial. The status indicator can show that a subject gave a correct response when given controlled guidance from a therapist, which is known in this context as a “prompt” (“P”), that a subject gave no response (“0”), that a subject gave a correct response without needing controlled guidance (“+”), or that a subject gave an incorrect response (“−”).

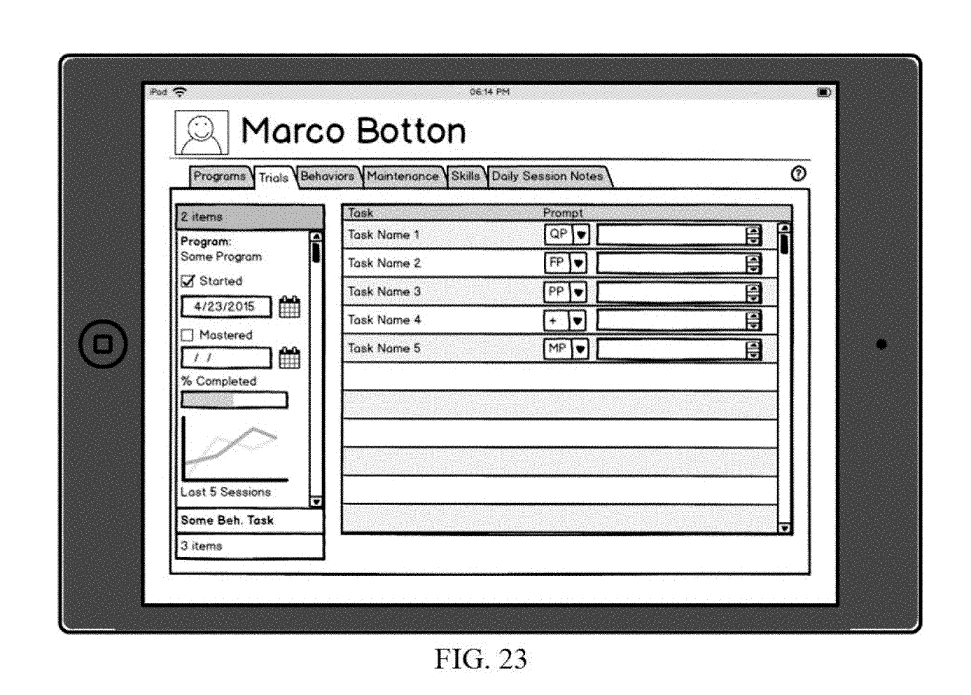

FIG. 23 shows a representative embodiment of the invention including a “Trials” display with information associated with a particular subject (a.k.a., “client”), where this display includes a grid containing information associated with tasks and prompts. Each line of the grid includes a task name, and a drop-down menu selector which can be used to enter data corresponding to a type of prompt which has been used in therapy for a task. Types of prompts that can be selected using this drop-down menu selector might include:+(Correct Response), VP (Vocal Prompt), GP (Gestural Prompt), PP (Partial Physical Prompt), FP (Full Physical Prompt), MP (Model Prompt), or PXP (Proximity Prompt).

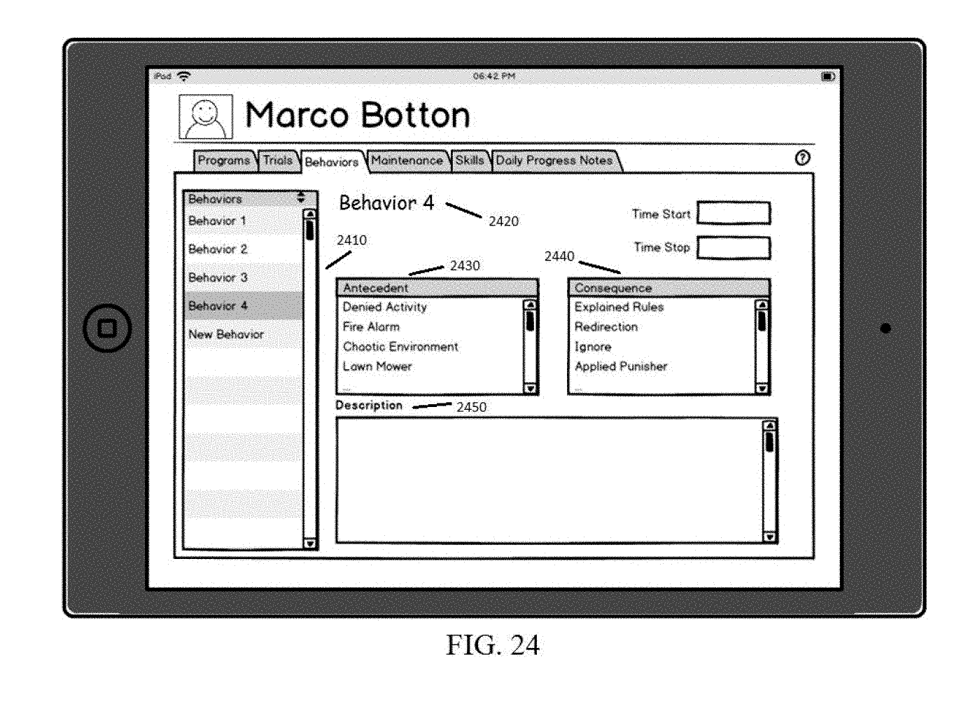

FIG. 24 shows a representative embodiment of the invention including a “Behaviors” display which is used in the creation or implementation of a behavior intervention plan (BIP). This display includes a behavior selector 2410, and a behavior title 2420 which appears when its corresponding behavior is selected. The display also includes an antecedent list 2430 and a consequence list 2440, which allow a therapist or other user to select data associated with a particular behavior. The display also includes a Description data entry field 2450, which is used to enter information describing details about a selected behavior.

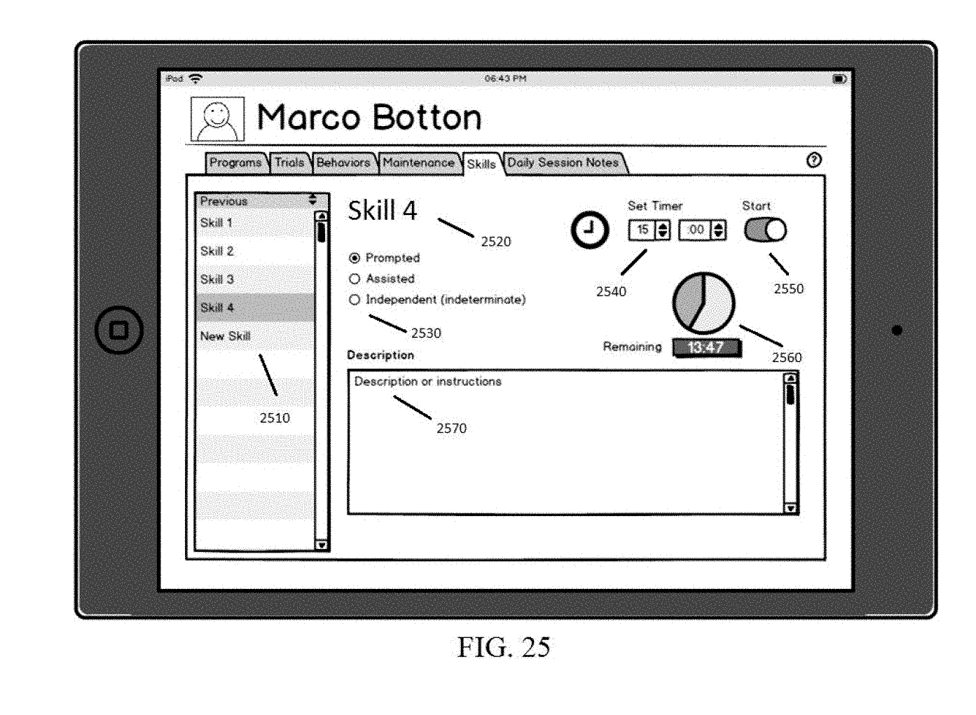

FIG. 25 shows a representative embodiment of the invention including a “Skills” display which is used in functional skills training for a subject; such training is often conducted during break periods in the middle of autism therapy sessions. This display includes a skill selector 2510, and a skill title 2520 which appears when its corresponding skill is selected. This display also includes a data selector 2530 which is used to select whether a skill has been completed in a prompted manner, an assisted manner, or an independent manner. This display also includes timer set boxes 2540 which can be used to select a time to be counted down, and a start switch 2550 which can start or stop a countdown timer 2560. The countdown timer 2560 can include a circle with “pie chart” style sections, where one colored section indicates time elapsed and a differently colored section indicates time remaining. The countdown timer 2560 can also include a numerical display indicating how much time remains. This display also includes a description field 2570 which can be used to enter a description or instructions relating to a selected skill.

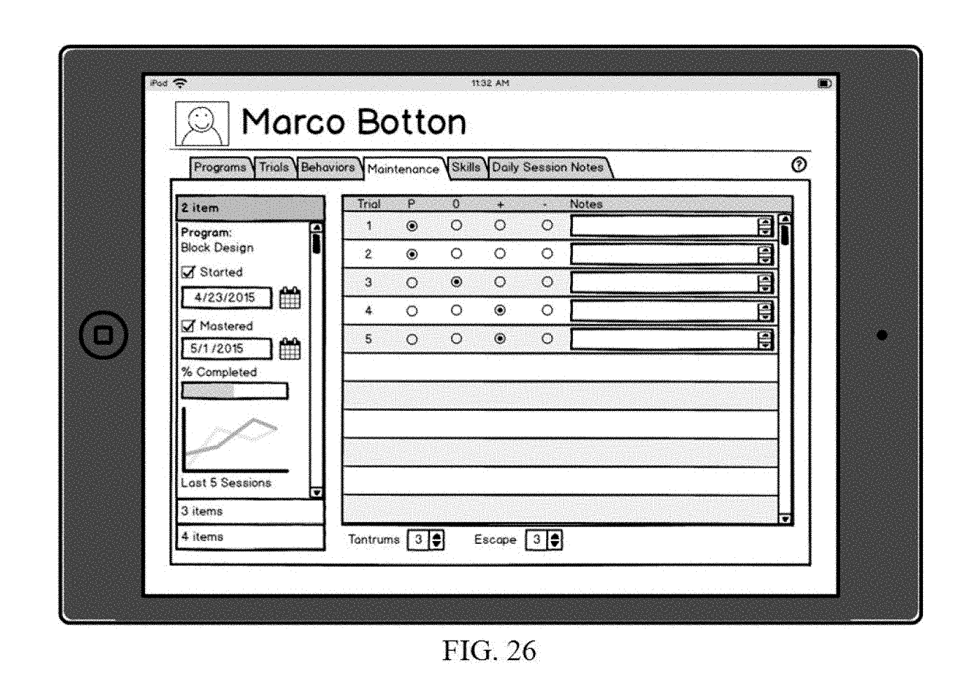

FIG. 26 shows a representative embodiment of the invention including a “Maintenance” display with information associated with a particular subject (a.k.a., “client”), where this display includes data from trials conducted in a maintenance process. In this context, a “maintenance process” refers to trials that are conducted with a therapist and a subject in which skills that have been previously learned by the subject are assessed and/or tested. The information on this display also includes a name of a program being worked on (“Block Design”, in this case), a start date, and a date showing when a particular skill was mastered. This display also includes a grid which lists trials that have been conducted, along with a selector which may be used to select a status indicator for each trial. The status indicator can show that a subject gave a correct response when given controlled guidance from a therapist, which is known in this context as a “prompt” (“P”), that a subject gave no response (“0”), that a subject gave a correct response without needing controlled guidance (“+”), or that a subject gave an incorrect response (“−”).

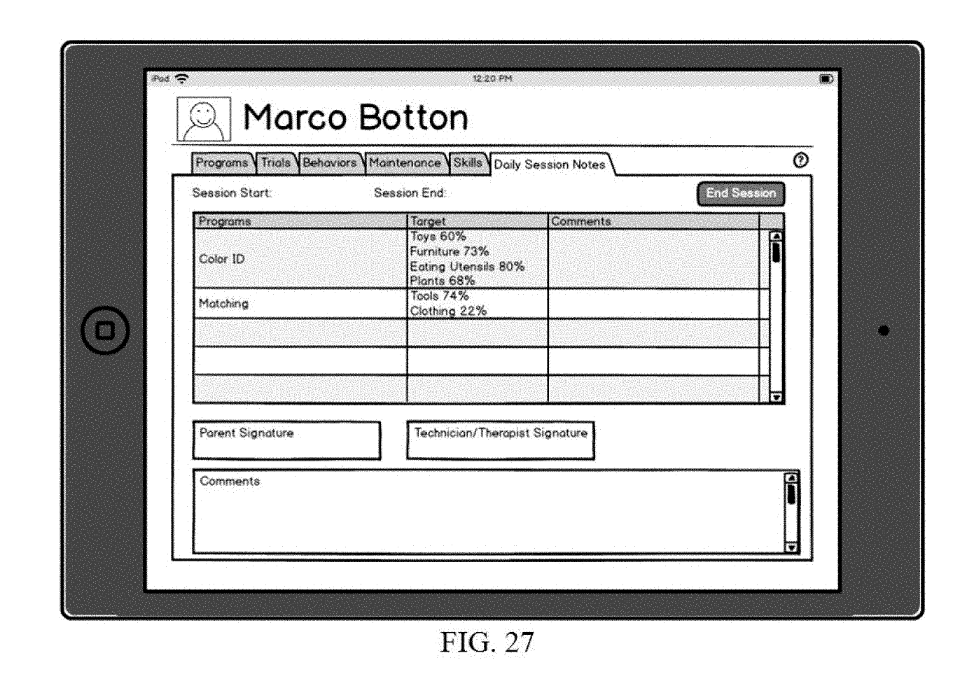

FIG. 27 shows a representative embodiment of the invention including a “Daily Session Notes” display with information associated with a particular subject (a.k.a., “client”). This display contains information which includes data collected during a therapy session. This display includes a grid in which programs are listed, along with associated targets and associated comments. Each target in the grid is listed with a number indicating a rate of completion or a percentage correct; this number was determined through therapy processes such as trials during a particular therapy session. This display also includes data fields for session start, session end, parent signature, and technician/therapist signature.

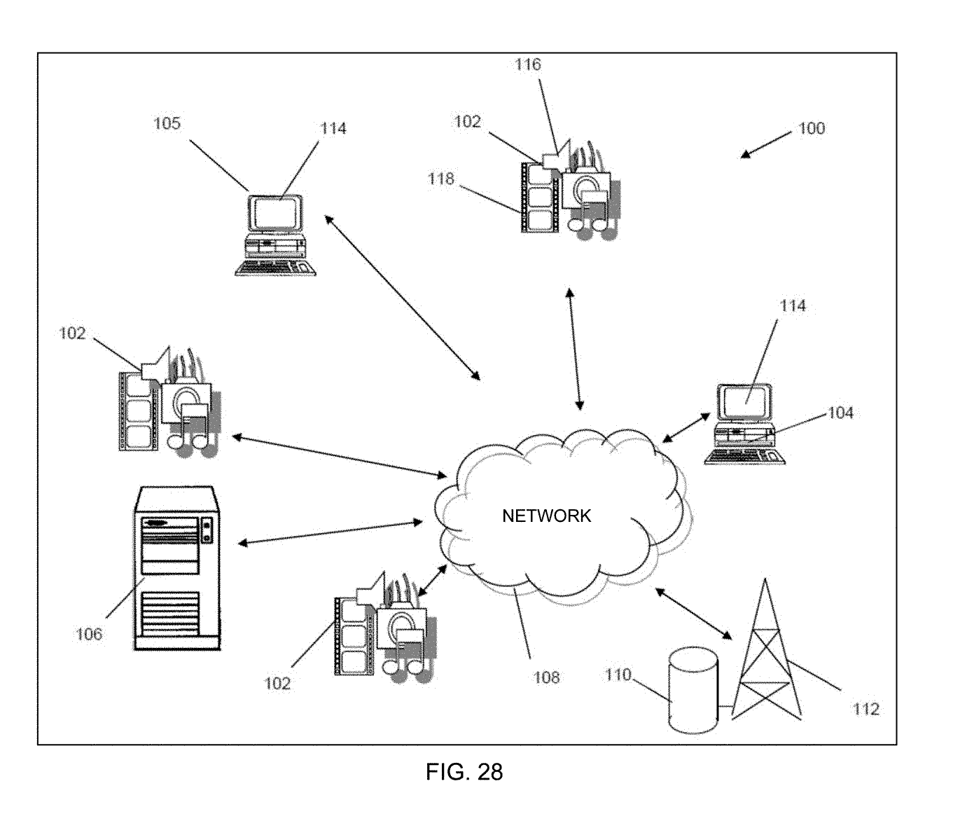

FIG. 28 is a schematic diagram illustrating an overall view of communication devices, computing devices, and mediums for implementing a system and method for automatically conducting tasks in support of autism therapy sessions for a human subject.

The system 100 includes multimedia devices 102 and desktop computer devices 104 configured with display capabilities 114 and processors for executing instructions and commands. The multimedia devices 102 are optionally mobile communication and entertainment devices, such as cellular phones, tablets, and mobile computing devices that in certain embodiments are wirelessly connected to a network 108. The multimedia devices 102 typically have video displays 118 and audio outputs 116. The multimedia devices 102 and desktop computer devices 104 are optionally configured with internal storage, software, and a graphical user interface (GUI) for carrying out elements of the autism therapy sessions platform according to embodiments of the invention. The network 108 is optionally any type of known network including a fixed wire line network, cable and fiber optics, over the air broadcasts, local area network (LAN), wide area network (WAN), global network (e.g., Internet), intranet, etc. with data/Internet capabilities as represented by server 106. Communication aspects of the network are represented by cellular base station 110 and antenna 112. In a preferred embodiment, the network 108 is a LAN and each remote device 102 and desktop device 104 executes a user interface application (e.g., Web browser) to contact the server system 106 through the network 108. Alternatively, the remote devices 102 and 104 may be implemented using a device programmed primarily for accessing network 108 such as a remote client.

The software for the autism therapy sessions platform, of embodiments of the invention, may be resident on tablets, 102 desktop or laptop computers 104, or stored within the server 106 or cellular base station 110 for download to an end user. Server 106 may implement a cloud-based service for implementing embodiments of the platform with a multi-tenant database for storage of separate client data for each autism treatment agency carried out on the platform.

The foregoing description is illustrative of particular inventive embodiments of the invention, but is not meant to be a limitation upon the practice thereof. The following claims, including all equivalents thereof, are intended to define the scope of the invention:

1. A method for using a computing device to automatically conduct tasks in support of autism therapy sessions for a human subject comprising:
creating an autism therapy plan by inputting the assessment results into a mathematical model;
implementing the autism therapy plan by conducting therapy programs;
monitoring therapy program results, and
creating a revised autism therapy plan which is generated using the therapy program results as an input.

2. The method of claim 1, wherein inputting and documenting results of the subject assessment comprises:
navigating to a portal using a web browser;
selecting a client from a client list within the portal;
selecting a user-interface element which is associated with the client;
opening a note element which is associated with the user interface element, and
populating the note element with information from an existing note element.

3. The method of claim 1, wherein creating an autism therapy plan further comprises one or more of:
identifying a number of required skills;
creating a program for each of the required skills;
identifying program goals;
identifying program targets;
identifying program instructions; and
identifying program mastery criteria.

4. The method of claim 1, wherein conducting a subject assessment comprises:
initiating a functional assessment;
selecting a selected assessment;
conducting the selected assessment; and
completing the functional assessment.

5. The method of claim 4, wherein the selected assessment is either a Verbal Behavior Milestones Assessment and Placement Program (VB-MAPP), or an Assessment of Basic Language and Learning Skills (ABLLS).

6. The method of claim 1, wherein implementing the autism therapy plan further comprises:
setting up data sheets;
initiating progress notes by listing programs and targets;
selecting certain selected programs to work on; and
completing the certain selected programs.

7. The method of claim 6, wherein completing the certain selected programs comprises:
running trials on new programs; and
completing maintenance on previously completed programs.

8. The method of claim 6, wherein completing the certain selected programs comprises:
selecting a target from a certain selected program;
conducting probes; and
determining whether a number of consecutive sessions have been completed.

9. The method of claim 6, wherein completing the certain selected programs comprises:
selecting a target from a certain selected program;
identifying probe criteria;
conducting probes; and
determining whether a number of consecutive sessions have been completed.

10. The method of claim 1, further comprising:
providing a notification prompt to take a break; and
recording a description of how a program was completed.

11. The method of claim 1, further comprising:
completing a behavior intervention plan;
completing progress notes by recording target percentages and problem behaviors;
documenting subject behaviors; and
closing out the autism therapy session.

12. The method of claim 1, wherein monitoring the autism therapy plan comprises:
determining behaviors for a behavior intervention plan.

13. The method of claim 1, wherein creating a revised autism therapy plan further comprises:
selecting programs, targets, or a combination thereof from the autism therapy plan;
determining whether or not at least one program, at least one target, or a combination thereof has been mastered;
electronically documenting mastered programs or mastered targets in a maintenance list;
electronically documenting non-mastered programs or non-mastered targets in an acquisition list;
calculating new programs, new targets, or a new combination thereof by using the maintenance list and the acquisition list as inputs to a mathematical model; and
integrating the new programs, new targets, or new combination into a revised autism therapy plan.

14. A method of using a computing device to on-board and assign clients and staff in support of autism services, said method comprising:
registering through the use of a web portal;
verifying payment information through the web portal;
creating an agency database;
providing a login link to an administrator;
accessing the agency database through the login link;
adding autism clients and staff to the agency database;
assigning staff to autism clients; and
creating an autism behavior therapy plan.

15. The method of claim 14, further comprising viewing a client's autism behavior therapy plan.

16. A method of using a mobile device to initiate autism therapy sessions, said method comprising:
logging in;
selecting a client;
selecting a program for use in autism therapy sessions with the client;
applying filters to the program;
viewing the program;
entering a planned session duration; and
starting a session.

17. The method of claim 16, further comprising applying target filters after viewing the program.

18. A method of using a mobile device to conduct trials in autism therapy sessions, said method comprising:
selecting a program;
selecting a target;
using logic to automatically determine if the selected target is in probe mode; and
if the logic determines that the selected target is in probe mode, entering or populating probe details, and recording probe details using electronic documentation; and
alternatively, if the logic determines that the selected target is not in probe mode, recording trials using electronic documentation.

19. A system comprising a computing device adapted to automatically conduct tasks in support of autism therapy sessions for a human subject, said tasks comprising:
creating an autism therapy plan by inputting the assessment results into a mathematical model;
implementing the autism therapy plan by conducting therapy programs;
monitoring therapy program results, and
creating a revised autism therapy plan which is generated using the therapy program results as an input.

20. The system of claim 19 further comprising:
conducting a subject assessment to generate assessment results relating to degrees of autism of the human subject; and
documenting the assessment results electronically prior to said creating said autism therapy plan.
